(19) United States
(12) Patent Application Publication
(10) Pub. No.: US 2018/0317935 A1
(43) Pub. Date: Nov. 8, 2018

(54) TOURNIQUET WITH AN ATTACHED TIMER

(71) Applicant: Rachel Ann Engwall, Fairfield, IA (US)

(72) Inventor: Rachel Ann Engwall, Fairfield, IA (US)

(21) Appl. No.: 15/588,834

(22) Filed: May 8, 2017

(51) Int. Cl. A61B 17/132

(52) U.S. Cl. CPC A61B 17/1327; A61B 2090/0807

(57) ABSTRACT

A tourniquet with an attached timer. The tourniquet includes a main strap. The main strap is an elongated strap having a first end opposite a second end, and an upper surface opposite a lower surface. The timer is attached to the main strap. The timer is used to keep track of the amount of time the tourniquet is applied to the user.

### BACKGROUND OF THE INVENTION

The present invention relates to tourniquets and, more particularly, to a tourniquet with a time keeper.

A tourniquet is a constricting or compressing device, specifically a bandage, used to control venous and arterial circulation to an extremity for a period of time. Pressure is applied circumferentially upon the skin and underlying tissues of a limb; this pressure is transferred to the walls of vessels, causing them to become temporarily occluded. It is generally used as a tool for a medical professional in applications such as cannulation or to stem the flow of traumatic bleeding, especially by military medics. The tourniquet is usually applied when the patient is in a life-threatening state as a result of continuous bleeding. It may be difficult to keep track of an amount of time that the tourniquet is applied to the patient in a stressful situation.

As can be seen, there is a need for an improved tourniquet that displays an amount of time the tourniquet is applied to the patient.

### SUMMARY OF THE INVENTION

In one aspect of the present invention, a tourniquet comprises: a main strap comprising a first end, a second end, an upper surface and a lower surface; a buckle connecting the first end to the second end forming a loop, wherein the main strap is adjustable within the buckle to change a diameter of the loop; a windlass strap comprising a first end and a second end each attached to the upper surface of the main strap forming a loop portion therebetween; a windlass rod secured to the loop portion of the windlass strap; and a timer attached to the strap, wherein the timer comprises a starter operable to initiate the timer when activated.

In another aspect of the present invention, a method of keeping time during a use of a tourniquet comprising the steps of: providing the tourniquet comprising: a main strap comprising a first end, a second end, an upper surface and a lower surface; and a timer attached to the main strap, wherein the timer comprises a starter operable to initiate the timer when activated; wrapping the strap around a limb of a user; connecting the first end and the second end of the strap together; tightening the strap against the limb; and initiating the timer by activating the starter.

These and other features, aspects and advantages of the present invention will become better understood with reference to the following drawings, description and claims.

### BRIEF DESCRIPTION OF THE DRAWINGS

FIG. 1 is a perspective view of an embodiment of the present invention shown in use;

FIG. 2 is a perspective view of an embodiment of the present invention;

FIG. 3 is an exploded view of an embodiment of the present invention; and

FIG. 4 is a section view of the present invention taken along line 4-4 in FIG. 1.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

The present invention includes a time calculating apparatus that is attached to a tourniquet. The timer attaches to the tourniquet or can be made as part of the tourniquet and continuously keeps track of the time the tourniquet was applied to the patient. The time keeping piece keeps track of how much time has passed from being activated which is when the tourniquet was applied. The time keeping piece does not stop which allows a continuous clock. The attachment piece keeps the time keeping piece secured to the tourniquet in a fashion that does not interfere with the tourniquets viability.

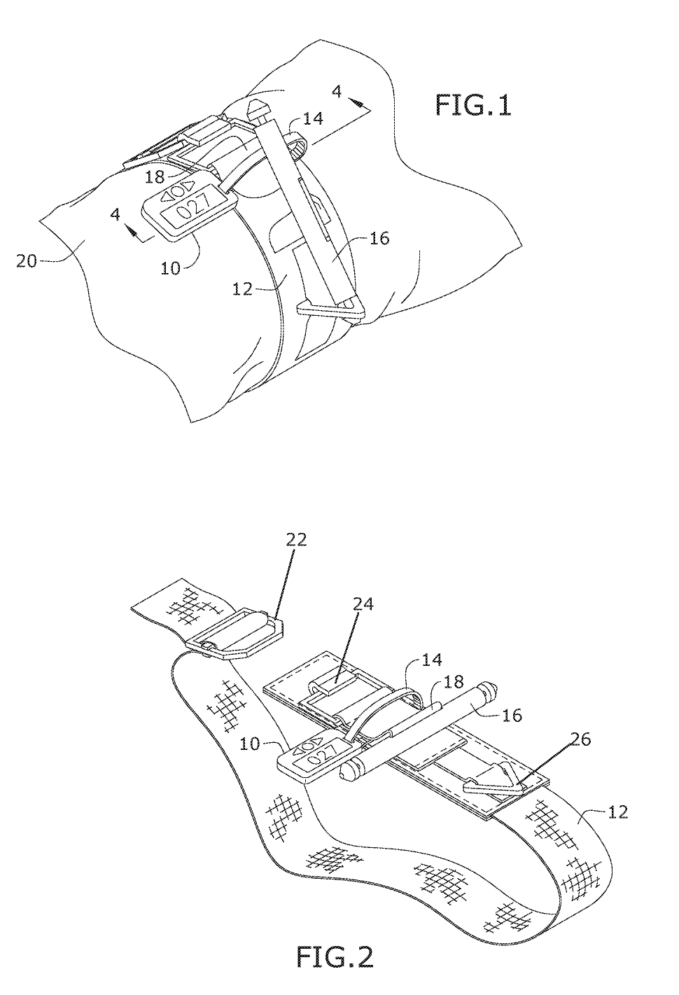

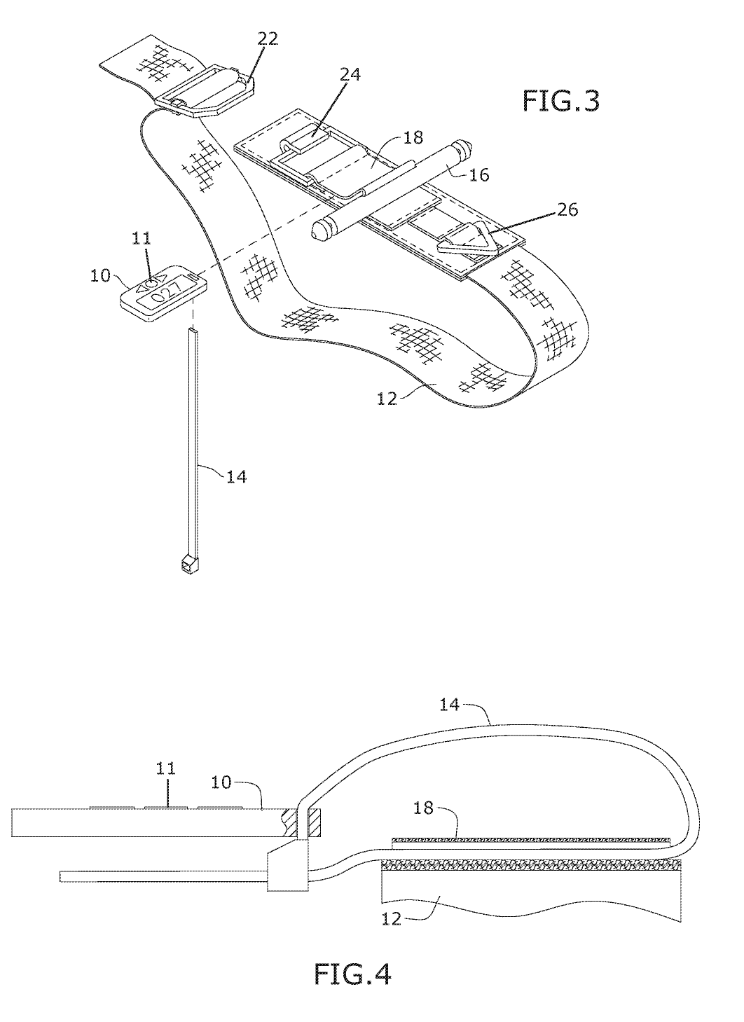

Referring to FIGS. 1 through 4, the present invention includes a tourniquet with an attached timer 10. The tourniquet includes a main strap 12. The main strap 12 is an elongated strap having a first end opposite a second end, and an upper surface opposite a lower surface. The main strap 12 may be made of a material having a rubber elasticity, such as rubber, silicone or other stretchable polymers. Alternatively, the main strap 12 may be made of a woven fabric. The timer 10 is attached to the main strap 12. The timer 10 is used to keep track of the amount of time the tourniquet is applied to the user 20.

The present invention may further include a buckle 22 connecting the first end of the main strap 12 to the second end forming a loop. The main strap 12 is adjustable within the buckle 22 to change a diameter of the loop. Therefore, a user may wrap the main strap 12 around a user's appendage and tighten the main strap 12 against the appendage using the buckle 22. The buckle 22 may include a frame and a transverse bar. The main strap 12 may wrap around the transverse bar. Alternatively, the buckle 22 may further include a pivoting prong that fits within apertures formed through the main strap 12. In certain embodiments, a hook 24 is secured to the upper surface of the main strap 12. The hook 24 releasably secures to the buckle 22.

The tourniquet may further utilize a windlass rod 16. In such embodiments, a windlass strap 18 is attached to the upper surface of the main strap 12. The windlass strap 18 includes a first end and a second end each attached to the upper surface of the main strap 12 forming a loop portion therebetween. The windless rod 16 may include an elongated solid rod. The windlass rod 16 is secured to the loop portion of the windlass strap 18. A looped clip 26 is secured to the upper surface of the main strap 12 and is sized to receive an end of the windlass rod 16. To further tighten the tourniquet to the user's limb, the windlass rod 16 is rotated until the bleeding stops. The end of the windless rod 16 is placed within the looped clip 26 to prevent the windlass rod 16 from unwinding.

As mentioned above, the timer 10 is attached to the main strap 12. The timer 10 includes a display showing an amount of time that has passed since the timer 10 was initiated. The timer 10 further includes a starter 11 operable to initiate the timer when activated. The starter 11 may be in the form of a button or a representation of a button on a touch screen. The timer 10 may be digital with a screen display. Alternatively, the timer 10 may be an analog timer. The timer 10 may be secured to the windlass strap 18 by a zip tie 14. Alternatively, the timer 10 may be stitched directly to the upper surface of the main strap 12.

A method of keeping time during a use of a tourniquet may include the following steps: providing the tourniquet described above; wrapping the strap of the tourniquet around a limb of a user; connecting the first end and the second end of the strap together; tightening the strap against the limb; and initiating the timer by activating the starter. The step of tightening the strap against the limb may include the steps of: reducing the diameter of the loop using the buckle; twisting the windlass rod until bleeding of the user stops; and inserting the end of the windlass rod into the clip.

It should be understood, of course, that the foregoing relates to exemplary embodiments of the invention and that modifications may be made without departing from the spirit and scope of the invention as set forth in the following claims.

What is claimed is:

1. A tourniquet comprising:
a main strap comprising a first end, a second end, an upper surface and a lower surface;
a buckle connecting the first end to the second end forming a loop, wherein the main strap is adjustable within the buckle to change a diameter of the loop;
a windlass strap comprising a first end and a second end each attached to the upper surface of the main strap forming a loop portion therebetween;
a windlass rod secured to the loop portion of the windlass strap; and
a timer attached to the strap, wherein the timer comprises a starter operable to initiate the timer when activated.

2. The tourniquet of claim 1, wherein the timer is a digital timer and the starter is a button.

3. The tourniquet of claim 1, further comprising a zip tie securing the timer to the windlass strap.

4. The tourniquet of claim 1, further comprising a clip secured to the upper surface of the main strap and sized to receive an end of the windlass rod.

5. The tourniquet of claim 1, further a hook secured to the upper surface of the main strap, wherein the hook releasably secures to the buckle.

6. A method of keeping time during a use of a tourniquet comprising the steps of:
providing the tourniquet comprising:
a main strap comprising a first end, a second end, an upper surface and a lower surface; and
a timer attached to the main strap, wherein the timer comprises a starter operable to initiate the timer when activated;
wrapping the strap around a limb of a user;
connecting the first end and the second end of the strap together;
tightening the strap against the limb; and
initiating the timer by activating the starter.

7. The method of claim 6, wherein the tourniquet further comprises:
a buckle connecting the first end to the second end forming a loop, wherein the main strap is adjustable within the buckle to change a diameter of the loop;
a windlass strap comprising a first end and a second end each attached to the upper surface of the main strap forming a loop portion therebetween;
a windlass rod secured to the loop portion of the windlass strap; and
a clip secured to the upper surface of the strap and sized to receive an end of the windlass rod.

8. The method of claim 7, wherein the step of tightening the strap against the limb comprises the steps of:
reducing the diameter of the loop using the buckle;
twisting the windlass rod until bleeding of the user stops; and
inserting the end of the windlass rod into the clip.

9. The method of claim 7, wherein the timer is a digital timer and the starter is a button.

10. The method of claim 7, further comprising a zip tie securing the timer to the windlass strap.
